(19) United States
(12) Patent Application Publication
(10) Pub. No.: US 2015/0098905 A1
(43) Pub. Date: Apr. 9, 2015

(54) METHODS TO DETECT A FUNGAL CELL

(71) Applicant: Rutgers, The State University of New Jersey, New Brunswick, NJ (US)

(72) Inventors: David S. Perlin, New York, NY (US); Guillermo Garcia-Effron, Newark, NJ (US); Arkady Mustaev, New York, NY (US)

(73) Assignee: Rutgers, The State University of New Jersey, New Brunswick, NJ (US)

(21) Appl. No.: 14/399,772

(22) PCT Filed: May 8, 2013
(86) PCT No.: PCT/US2013/040182

§ 371 Date: Nov. 7, 2014

Related U.S. Application Data
(60) Provisional application No. 61/644,283, filed on May 8, 2012.

(51) Int. Cl. A61K 49/00

(52) U.S. Cl. CPC A61K 49/0056; A61K 49/0052; A61K 49/0026; A61K 49/0021
USPC 424/9.6; 530/317; 544/361; 435/7.31

(57) ABSTRACT

The present invention relates to targeting agents and methods: of using the targeting agents to detect a fungal cell in a subject.

### CROSS REFERENCE TO RELATED APPLICATION

This application claims the benefit of U.S. Provisional Application No. 61/644,283 filed May 8, 2012, the disclosure of which is incorporated herein by reference in its entirety.

### STATEMENT REGARDING FEDERALLY SPONSORED RESEARCH

This work was supported by a grant from the National Institutes of Health (AI069397). The U.S. Government has certain rights in this invention.

### FIELD OF THE INVENTION

The present invention relates to targeting agents and methods of using the targeting agents to detect a fungal cell in a subject.

### BACKGROUND OF THE INVENTION

Invasive fungal infections (IFI) are a growing threat to human health due to both immunocompromising diseases and chronic infections. In most situations where IFI diagnosis is considered, the clinical presentation is often non-specific and can be caused by a wide range of infectious organisms, underlying illness, or complications of treatment. Successful IFI diagnosis is further complicated due to uncertainties and controversies in disease definition and in selecting standardized methods for establishing the diagnosis. Fungal cell wall components such as glucans and galactomannans, which are actively shed during growth and development, are the basis for biomarker-based commercial antigen assays for rapid diagnostic testing, but their value is limited by the potential for false-positive and false-negative results due to an assortment of factors. Imaging is an important part of the diagnosis of diseases, such as invasive aspergillosis (IA). Characteristic images from conventional X-rays and more advanced computed tomography (CT) can be used to identify disease lesions in neutropenic patients and help manage IA. However, diagnostic imaging is inherently non-specific and is dependent on other clinical signs and symptoms. There is a need for a broad-spectrum fungal-specific targeting molecule with a label to selectively detect invasive fungal infections in a subject.

### SUMMARY OF THE INVENTION

The present invention relates to targeting agents and methods of using the targeting agents to detect a fungal cell in a subject. The present invention fulfills the need for methods of detecting fungus in a patient using a broad-spectrum fungal-specific targeting molecule.

In one aspect, the present invention provides a method to detect a fungus in a subject comprising administering to said subject a targeting agent wherein said targeting agent comprises an antifungal drug covalently bound to a detectable label, and detecting said targeting agent. The detectable label may be a fluorescent label, a radioactive isotope, or a contrast agent. The fluorescent label may be boron-dipyrromethene (BODIPY), 7-hydroxy-9H-(1,3-dichloro-9,9-dimethylacridin-2-one)(DDAO), 7-amino-9H-(1,3-dichloro-9,9-dimethylacridin-2-one)(7-aminoDDAO), or a derivative thereof. The antifungal drug may be a polyene, an azole and an echinocandin. The antifungal drug may be natamycin, rimocidin, filipin, nystatin, amphotericin B, candicin, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, abalungin, terbinafine, amorolfine, naftifine, butenafine, anidulafungin, caspofungin, and micafungin. The targeting agent may be caspofungin-7aminoDDAO.

The subject may also be administered a pretreatment antifungal drug prior to the administration of said targeting agent, wherein said pretreatment antifungal drug and said the antifungal drug of said targeting agent are the same antifungal drug, e.g. caspofungin.

The subject may also be administered a pretreatment antifungal drug prior to the administration of said targeting agent, wherein said pretreatment antifungal drug and the antifungal drug in said targeting agent are not the same antifungal drug and wherein said pretreatment antifungal drug does not bind to the same target as the antifungal drug in said targeting agent, e.g. the pretreatment antifungal drug is posaconazole, and the antifungal drug in said targeting agent is caspofungin.

The targeting agent may be detected using an imaging device, including without limitation an x-ray imaging device, an infrared imaging device, fluorescent imaging device, nuclear magnetic resonance imaging device, magnetic resonance spectroscopy device, and a positron emission tomography device. The fungus that may be detected includes without limitation, Candida albicans, Candida glabrata, Candida parapsilosis, Candida krusei, Aspergillus fumigatus, Aspergillus niger, Aspergillus flavus, Cryptococcus neoformans, Scedosporium apiospermum, Zygomycetes, Histoplasma capsulatum, Coccidioides immitis, Paracoccidiioides brassiliensis and Blastomyces dermatitidis.

In a second aspect, the present invention provides a targeting agent comprising an antifungal agent conjugated directly to a detectable label. The detectable label may be a fluorescent label, and the antifungal agent may be caspofungin or posacozole. The detectable label may be boron-dipyrromethene, 7-hydroxy-9H-(1,3-dichloro-9,9-dimethylacridin-2-one), 7-amino-9H-(1,3-dichloro-9,9-dimethylacridin-2-one) or a derivative thereof.

In a third aspect, the present invention provides a kit for detecting a fungus in a biological sample or a subject comprising the targeting agent as described, and instructions for use.

### BRIEF DESCRIPTION OF THE DRAWINGS

FIG. 1 illustrates the chemical structure for a derivative of caspofungin (CSF) covalently attached to boron-dipyrromethene (BODIPY) and amine attachment sites are circled.

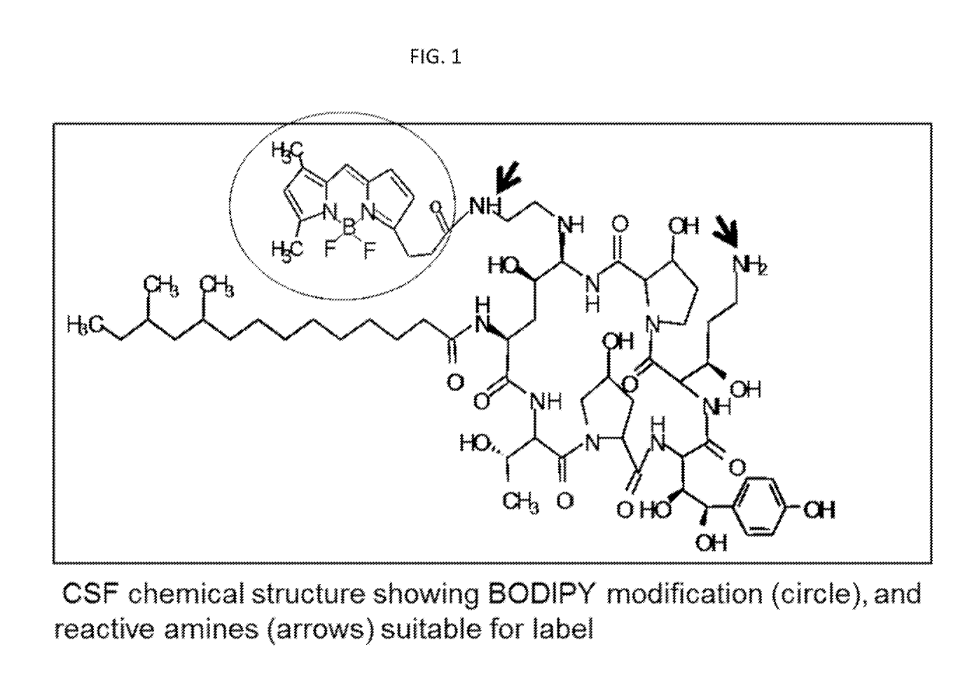

FIG. 2 depicts the synthesis and light emission properties of 7-hydroxy-9H-(1,3-dichloro-9,9-dimethylacridin-2-one) (DDAO), 7-aminoDDAO and its derivatives.

FIG. 3 depicts caspofungin (CSF) covalently bound to 7-aminoDDAO and posaconazole (POS) covalently bound to 7-aminoDDAO.

FIG. 4 depicts the synthetic steps to covalently attach 7-aminoDDAO to POS.

FIG. 5 depicts the synthetic steps to covalently attach BODIPY to POS (POS-BOD).

FIG. 6 depicts the chemical structure of posaconazole (POS), the attachment site for a label and POS covalently attached to BODIPY (POS-BOD).

### DETAILED DESCRIPTION OF THE INVENTION

The invention relates to targeting agents and the use of targeting agents to detect fungus in a subject.

### Targeting Agent and Methods of Use

In one embodiment, the present invention provides a targeting agent comprising an antifungal drug covalently bound to a detectable label. The antifungal agent may be a polyene, an azole, or an echinocandin. Examples of a polyene include hamycin, natamycin, rimocidin, filipin, nystatin, amphotericin B, and candicin. Examples of an azole include miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, and terconazole. Examples of an echinocandin include anidulafungin, caspofungin, and micafungin. In a further embodiment the antifungal drug specifically binds to an antifungal target and not to a target that is biologically produced in a mammal. The fungal target may be a carbohydrate, a peptide, a lipid or a combination thereof that is of fungal and not mammalian origin, e.g. beta (1,3) glucan synthase. In a preferred embodiment the targeting agent comprises caspofungin, posaconazole or a derivative thereof as known in the art.

The detectable label may be a fluorescent label, a radioactive isotope, or a contrast agent. In certain embodiments an antifungal drug is labeled with a radioactive isotope such as astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, hydrogen, iodine, iodine, iodine, indium, iron, phosphorus, rhenium, rhenium, selenium, ³⁵sulphur, technicium^{99m} and yttrium⁹⁰, ¹²⁵I, technicium^{99ml} and indium¹. Methods are known in the art to incorporate and covalently attach a selected radioactive isotope to an antifungal agent.

As used herein, terms “fluorophore,” “fluorescent moiety,” “fluorescent label,” “fluorescent dye” and “fluorescent labeling moiety” are used herein interchangeably. They refer to a molecule that, in solution and upon excitation with light of appropriate wavelength, emits light back. Numerous fluorescent labels of a wide variety of structures and characteristics are suitable for use in the practice of this invention. Similarly, methods and materials are known for fluorescently labeling a molecule of interest (see, for example, R. P. Haugland, “Molecular Probes: Handbook of Fluorescent Probes and Research Chemicals 1992-1994”, 5.sup.th Ed., a 1994, Molecular Probes, Inc.). In choosing a fluorescent label, it is often desirable that the fluorescent label absorbs light and emits fluorescence with high efficiency (i.e., high molar absorption coefficient and fluorescence quantum yield, respectively) and is photostable (i.e., it does not undergo significant degradation upon light excitation within the time necessary to perform the analysis). Furthermore in choosing a label it is preferred that the fluorescent label is (1) is small, e.g. (FW=294); (2) has a long-wavelength emission; (3) has fair brightness; (4) is pH-independent; (5) emission maximum up to about 680 nm, where the body tissues are the most transparent. In a preferred embodiment of the present invention, the fluorescent label is 7-hydroxy-9H-(1,3-dichloro-9,9-dimethylacridin-2-one) (DDAO), 7-amino-9H-(1,3-dichloro-9,9-dimethylacridin-2-one)(7-aminoDDAO), or a derivative thereof. DDAO derivatives may be used for covalent labeling of the biomolecule of interest, such as a targeting agent. DDAO derivatives contain an amine or amino group at position 7 instead of a hydroxyl group. In a preferred embodiment the hydroxyl group is replaced with the following formula NH—(CH2)X—NHY, wherein X=1-10 and Y═H, C, an alkyl, CS, (CH2)X may also be replaced with another spacer or polymer such as polyethylene glycol or other polymers that have the same properties and length. A DDAO fluorophore that contains an amino group at position 7 is herein referred to as 7-aminoDDAO. The synthetic intermediate 7-(4-aminobutyl)aminoDDAO can be easily converted to other reactive forms (e.g. thiol-, or click-reactive), which are useful for bioconjugation, methods are known in the art. DDAO derivatives that are 1.4-2.3 fold brighter then original DDAO. In a further embodiment the fluorescent label is boron-dipyrromethene (BODIPY) or a derivative thereof.

In certain embodiments, the antifungal drug is labeled with a contrast agent such as a paramagnetic metal ion which is used for Magnetic Resonance Imaging (MRI). Examples of such paramagnetic metal ions include, but are not limited to, gadolinium III (Gd3+), chromium 111 (Cr3+), dysprosium III (Dy3+), iron 111 (Fe3+), manganese II (Mn2+), and ytterbium III (Yb34+). Gadolinium is an FDA-approved contrast agent for MRI, and is known to provide great contrast between normal and abnormal tissues in different areas of the body.

The antifungal drug is covalently bound to the detectable label by methods known in the art and such that the resulting targeting agent maintains the specificity and sensitivity for the target of the antifungal agent.

In a further embodiment, targeting agents of the present invention may be formulated as a pharmaceutical composition, and may be administered to a mammalian host, such as a human patient, in a variety of forms adapted to the chosen route of administration, i.e., orally or parenterally, by intravenous, intramuscular, topical, subcutaneous, or other routes. Thus, the pharmaceutical composition of the invention may be systemically administered, e.g., orally, in combination with a pharmaceutically acceptable vehicle such as an inert diluent. They may be incorporated directly with the food of the patient's diet. For oral therapeutic administration, the compositions of the invention may be used in the form of elixirs, syrups, and the like. Any material used in preparing any unit dosage form should be pharmaceutically acceptable and substantially non-toxic in the amounts employed. To administer the pharmaceutical composition to a subject, it is preferable to formulate the molecules in a composition comprising one or more pharmaceutically acceptable carriers.

“Pharmaceutically acceptable carriers” include any and all clinically useful solvents, dispersion media, coatings, antibacterial and antifungal agents, isotonic and absorption delaying agents and the like. However, other solvents may also be employed. Under ordinary conditions of storage and use, these preparations may contain a preservative to prevent the growth of microorganisms, and other formulation ingredients as is known in the art.

The present invention further provides a method to detect a fungus in a subject by administering to a subject a targeting agent wherein the targeting agent comprises an antifungal drug covalently bound to a detectable label, and followed by detecting the targeting agent with an imaging device. The term “fungus” refers to fungal cells and related fungal structures that the targeting agent binds to e.g. glucan synthase. The targeting agent of the present invention can be administered to a subject by any of a number of means known in the art.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model. Typically, the terms “subject” and “patient” are used interchangeably herein in reference to a human subject.

The targeting agent of the invention may be administered in a variety of forms adapted to the chosen route of administration, i.e., orally or parenterally, by intravenous, intramuscular, topical, subcutaneous, or other routes, and then. Solutions may be prepared, for example, in water and/or with a pharmaceutically acceptable carrier.

In another embodiment, the present invention provides a method with an additional step wherein a pretreatment antifungal drug is administered to a subject prior to the administration of the targeting agent. In a preferred embodiment, the pretreatment antifungal drug and the antifungal drug of the targeting agent are the same antifungal drug. In a preferred embodiment, the pretreatment antifungal drug is caspofungin and the antifungal drug of the targeting agent is also caspofungin. In a further embodiment, the pretreatment antifungal drug and the antifungal drug of the targeting agent are not the same antifungal drug and the pretreatment antifungal drug does not bind to the same target as the antifungal drug of the targeting agent. For illustration purposes, the pretreatment antifungal drug may be posaconazole and the antifungal drug of the targeting agent may be caspofungin, and can be selected by one with ordinary skill in the art.

In certain embodiments, the imaging device to detect the targeting agent is a magnetic imaging device, an x-ray imaging device, an infrared imaging device, a fluorescent imaging device, nuclear magnetic resonance imaging device, magnetic resonance spectroscopy device, and a positron emission tomography device. One with ordinary skill in the art will adapt the proper modality to detect the targeting agent as described.

The present invention provides a broad spectrum targeting agent to detect a variety of fungi. The type of fungus that may be detected includes but is not limited to Candida albicans, Candida glabrata, Candida parapsilosis, Aspergillus fumigatus, Aspergillus niger, Aspergillus flavus, Cryptococcus neoformans, Scedosporium apiospermum, Zygomycetes, Histoplasma capsulatum, Coccidioides immitis, Paracoccidiioides brassiliensis or Blastomyces dermatitidis. Furthermore, one with ordinary skill in the art can determine the type of tissue, organ, or body fluid of a subject to detect, e.g. lungs, kidneys, sputum, BAL, blood, serum or urine.

In another embodiment, the present invention provides a kit for detecting a fungus in a biological sample or a subject comprising a targeting agent as previously described and instructions for use. “Biological sample” as used herein means a sample of biological tissue or fluid. Such samples include, but are not limited to, tissue isolated from animals. Biological samples may also include sections of tissues such as biopsy and autopsy samples, frozen sections taken for histologic purposes, blood, plasma, serum, sputum, saliva, stool, tears, mucus, hair, and skin. Biological samples also include explants and primary and/or transformed cell cultures derived from patient tissues. A biological sample may be provided by removing a sample of cells from an animal, but can also be accomplished by using previously isolated cells (e.g., isolated by another person, at another time, and/or for another purpose).

### Examples

The invention now being generally described, it will be more readily understood by reference to the following examples, which are included merely for purposes of illustration of certain aspects and embodiments of the present invention, and are not intended to limit the invention.

### Methods and Materials

Synthesis of BODIPY-Labeled Drugs.

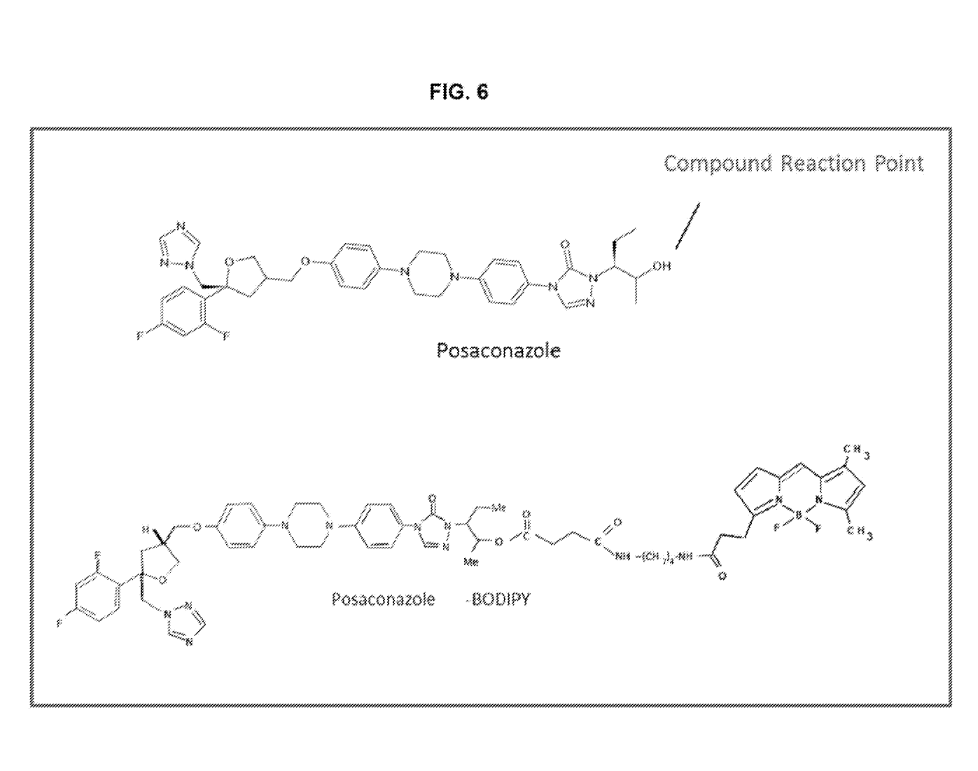

A derivative of CSF using BODIPY (BOD), an analog that resembles fluorescein but is smaller and more hydrophobic, was produced to test the CSF-BOD with various fungal pathogens. BODIPY-succinimidate was incubated with pure CSF in the presence of triethylamine as proton acceptor in DMF. The crude product was purified by TLC silica gel chromatography and characterized using mass spectroscopy, fluorescence and UV spectroscopy. The modified agent retained its specificity and sensitivity to the fungal target. To test these properties, the antifungal activity of the modified and un-modified root compound was evaluated and found to be effectively unaltered by the presence of label (C. albicans MICunlabeled=0.06 μg/ml v. MIClabeled=0.12 μg/ml) confirming that it retained its inherent potency. BODIPY coupled to posaconazole (POS) was formed by modifying a single hydroxyl group of posaconazole with succinic anhydride (FIG. 6).

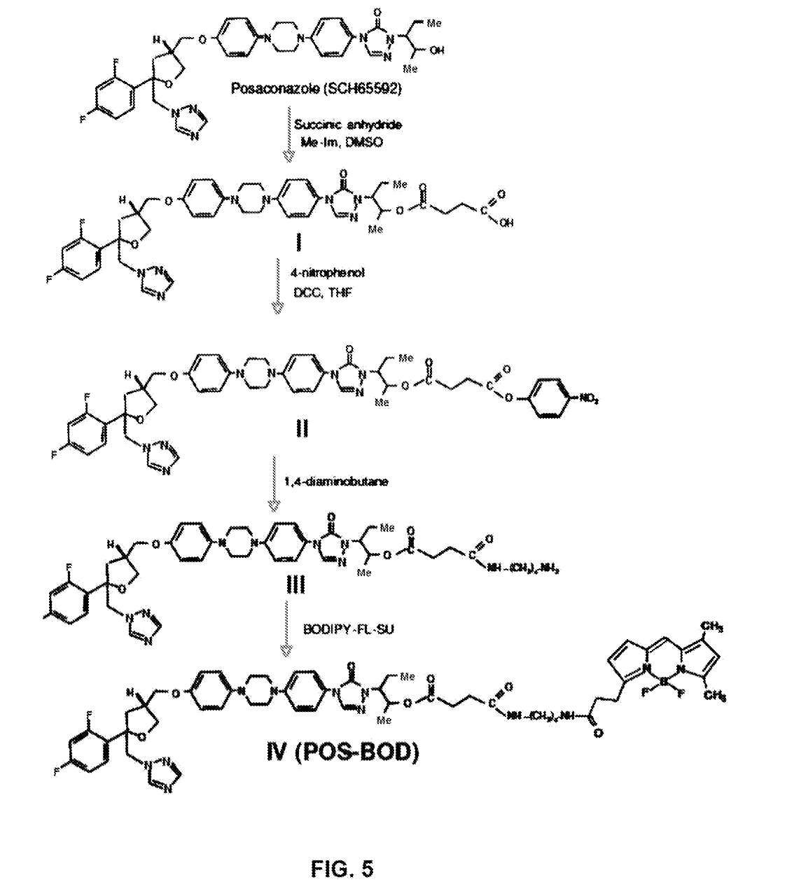

Incubation of the acylation product with 4-nitrophenol in the presence of DCC yielded a new compound consistent with the formation of an activated ester (FIG. 5, compound II). Incubation of this compound with ethylenediamine resulted in a characteristic absorption at 405 nm of nitrophenolate anion, which was indicative for acylation of the diamine by posaconazole activated ester. Incubation of the ester with aminobutane derivative of BODIPY fluorophore (FIG. 5, compound III) yielded fluorescent posaconazole-BODIPY adduct IV, with light absorption spectrum.

Cell Labeling.

To illustrate the potential of CSF-BOD and POS-BOD for visualizing fungal cells, the reagent was used to probe for the presence of Candida and Aspergillus species in a variety of matrices including solid and liquid growth media. The clinical A. fumigatus wild type strain R21 and Candida albicans ATCC strain 90028 were used for all the experiments. For Aspergillus, one drop of yeast extract peptone dextrose (YPD) agar was placed in the upper right corner of each well of a 15-well multitest slide followed by the addition of 10 μl of saline containing 105 conidia of R21. The slide was placed in a sterile petri dish with distilled water to provide a moist environment and incubated in a 37° C. incubator for 10-16 hours to facilitate germination and growth of hyphal elements. A 10 μl aliquot of CSF-BOD (170 ng/ml) or POS-BOD (150 ng/ml) was added to each well and incubated for 6 h at 37° C., followed by washing 3 times with sterile water and drying by vacuum. For Candida, an overnight culture of C. albicans was grown, washed by centrifugation and resuspended in dH2O. The yeast cells were added to RPMI and incubated at 200 rpm for 1 h at 37° C. to form germ tubes before being washed and resuspended in a 1 ml solution of CSF-BOD (120 ng/mil) or POS-BOD (150 ng/ml). The cells with drug were incubated again at 200 rpm for 1 h at 37° C., washed and resuspended in 50 ml of dH₂O. A 15 well slide was prepared using a poly-L-lysine to tightly adhere cells to the wells. A 10 μL aliquot of Candida cells was placed on each well and incubated for 10 minutes, aspirated off, and 1 μL Slow Fade Antifade reagent was added to prolong the fluorescent life and moisture level of the cells on the each slide. Each slide was observed under 100× magnification with the total internal reflection objective lens (TIRF) of a Nikon Eclipse 90i fluorescent microscope. The 15 wells of the slides were examined individually using Volocity 3D Image Analysis Software (PerkinElmer). Individual cells, hyphal elements and clusters of cells were visualized, analyzed, and captured in bright field lighting and repeated under a Green Fluorescent Protein (GFP) light setting.

### Results

Incubation of C. albicans at the MIC (120 ng/ml) for 1 h at 37° C. resulted in generalized fluorescence in the mother cell membrane with slightly more defined punctate fluorescence along the germ tube axis toward the growing tip, consistent with the putative intracellular vesicle trafficking of glucan synthase from clustered golgi vesicle complexes. Under 6 h and 37° C. conditions, A. fumigatus showed bright fluorescence in the spore but more diffuse labeling of the surface of the hyphal elements toward the growing apex consistent with a membrane location for glucan synthase.

Labeling was highly temperature sensitive with maximal labeling over the 6 h period observed at 37° C. Caspofungin and BODIPY alone failed to produce any labeling. The level of binding was greatly reduced in a well-characterized fks1-S645F mutant, which has reduced sensitivity of glucan synthase to echinocandins consistent with the probe binding to its intended target.

Representative Gram-negative and Gram-positive bacteria: Pseudomonas aeruginosa, Klebsiella pneumoniae, Streptococcus pneumoniae, Serratia marcescens, Staphylococcus aureus and Escherichia coli were grown and labeled under the same conditions. No fluorescence was observed in any cells.

Under the same labeling conditions as Caspofungin, POS-BOD with both Candida and Aspergillus showed generalized fluorescence labeling of mother cell and elongating hyphal elements. Pretreatment with unlabeled posaconazole or voriconazole greatly diminished or eliminated the fluorescence signal, while pretreatment with caspofungin had little effect on labeling of cells by POS-BOD.

Pretreatment of cells (C. albicans 1 h and A. fumigatus 2 h) at one dilution below the MIC with four different azoles, voriconazole (Pfizer), itraconazole (Janssen), posaconazole (Merck) and fluconazole (Pfizer), followed by CSF-BOD labeling described above had no effect on labeling. All samples showed the same fluorescence intensity with and without pretreatment consistent with azoles binding to a separate intracellular target. However, cells pretreated with the echinocandins, anidulafungin and micafungin, prior to standard labeling with the CSF-BOD probe eliminated the labeling. Pretreatment with caspofungin intensified the fluorescence.

The Synthesis of DDAO Fluorescent Derivatives.

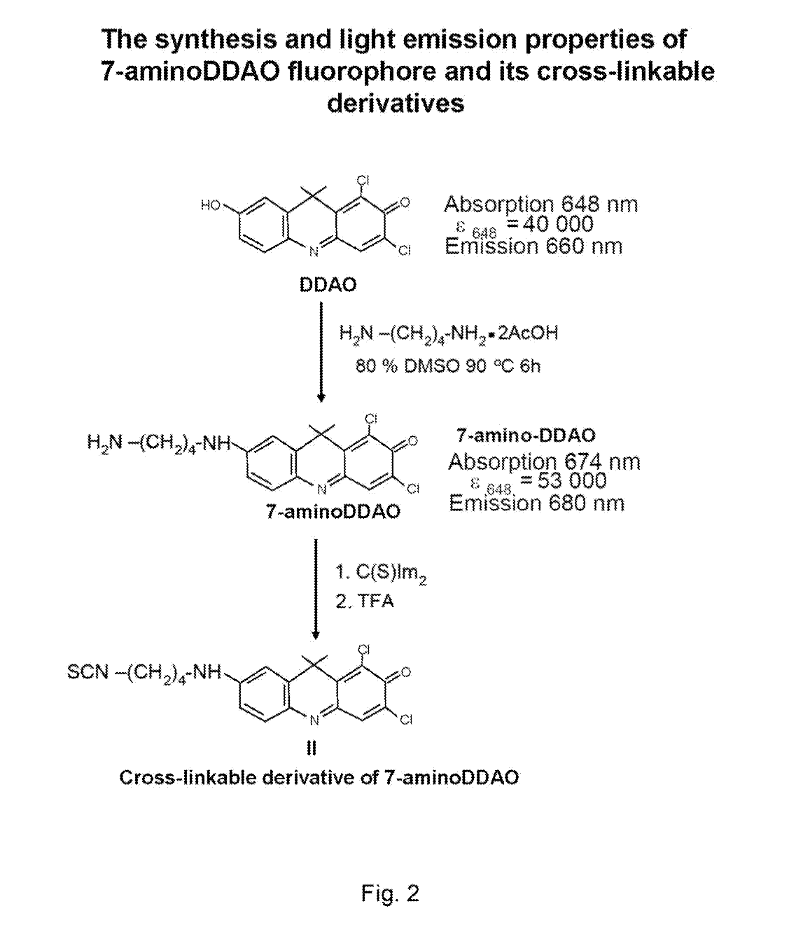

DDAO-NH—(CH2)4-NH2 (FIG. 2).

10 mg DDAO (7-hydroxy-9H (1,3-dichloro-9,9-dimethyl acridin-2-one)) (33 μmol) was dissolved in 100 μl 1 M diaminobutane diacetate in 80% aqueous DMSO. TLC analysis in acetonitrile-water (14:1) developing system detected intense-blue colored product migrating lower (Rf=0.45) than the original product (Rf=0.9). After 10 h incubation at 95° C. the reaction mixture was supplemented with 2 ml of water and extracted with ethylacetate (3×5 ml). The pH of the water layer was adjusted to 11-11.5 by 10 M KOH followed by extraction with ethylacetate (2×5 ml). The organic layer was collected and evaporated to dryness under reduced pressure affording 4 mg of compound I. UV λmax=(ε=M-1 cm-1), λmin=(ε=M-1 cm-1). MS: DDAO-NH—(CH2)4-NH2 (+1) 378.0887 (found) 378.288 (calculated).

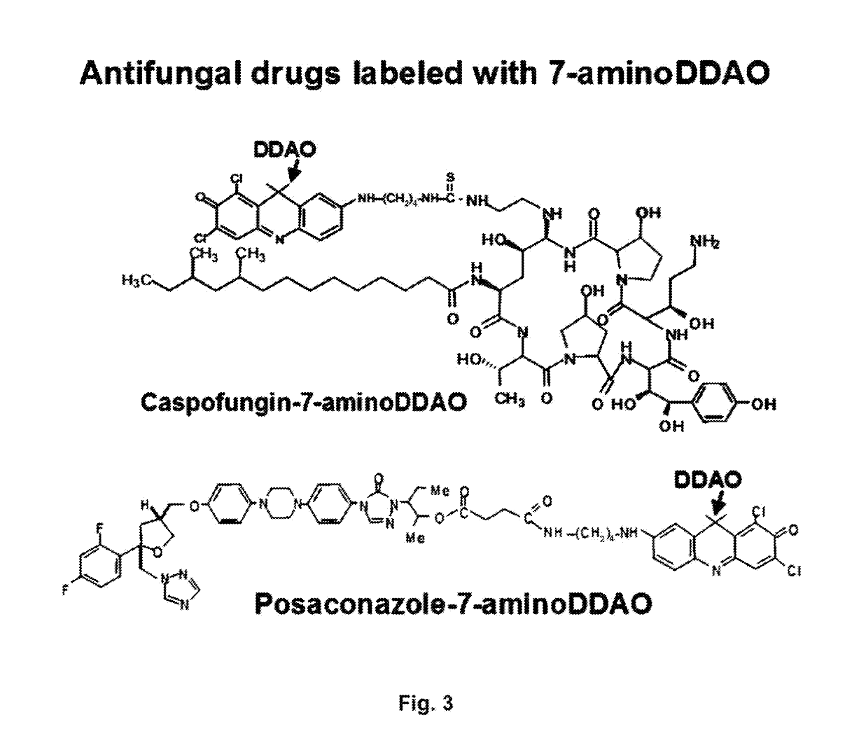

Caspofungin-DDAO Derivative (FIG. 3).

Caspofungin (2.6 mg, 2 μmol) was dissolved in the solution of 230 μl of 5 mM DDAO-NH—(CH2)4-NCS in DMF and 0.5 μl of TEA was added followed by incubation at 60° C. for 90 min. TLC in acetonitrile-water (5:1) developing system detected a blue-colored reaction product with Rf=0.65. Rfs for caspofungin and DDAO-NH—(CH2)4-NCS were 0.48 and 1.0 respectively. The product was purified by preparative TLC in acetonitrile-water (7:1) developing system, eluted by 50% aqueous methanol and the solution evaporated under reduced pressure to final concentration 0.33 mM. UV λmax=(ε=M-1 cm-1), λmin=(ε=M-1 cm-1), DDAO-NH2-(CH2)4-NCS-Caspofungin(+H) 1515.7242 (found) 1515.673 (calculated).

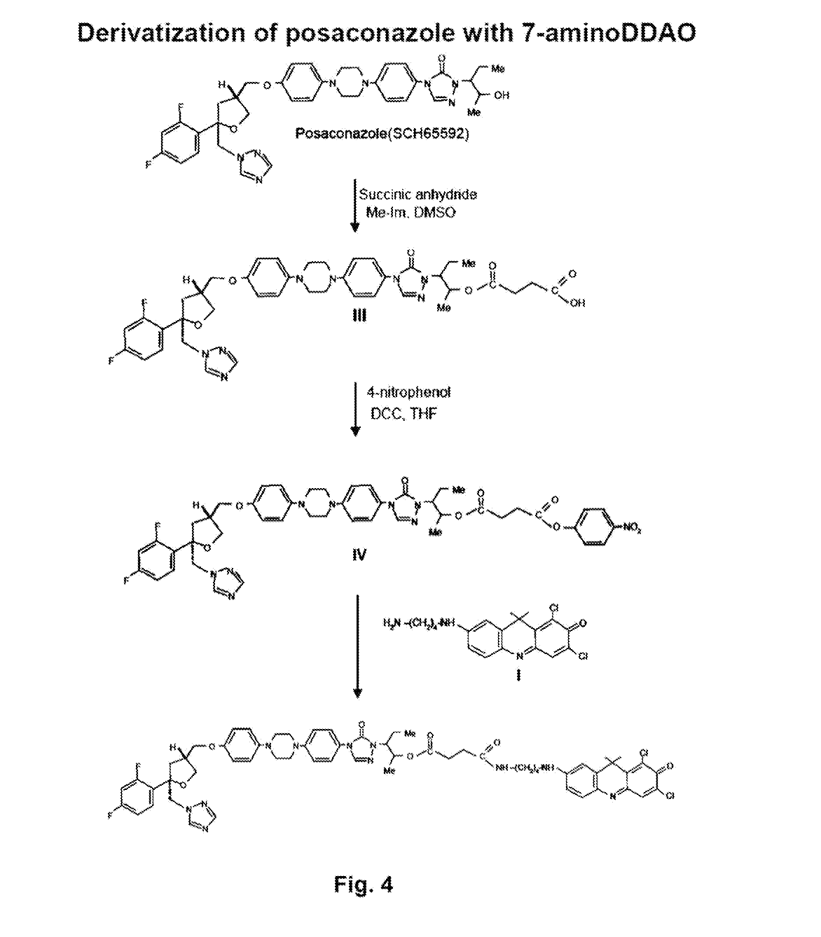

The Synthesis of Posaconazole-DDAO Derivative (FIG. 4).

Two milligrams of compound III (FIG. 4) were dissolved in 0.1 ml of 20 mM solution of compound I. The mixture was supplemented with 2 ml of triethylamine and left for 20 min at room temperature. TLC analysis in ethylacetate-ethanol (8:1) developing mixture revealed complete conversion of compound I to reaction product. The mixture was diluted by 2 ml of water, the residue collected by centrifugation, dissolved in DMF and subjected to preparative TLC in the same system. Yield 0.5 μmol.

To derivatize core DDAO compound Hamilton reaction previously discovered with simpler phenol-, or naphtol-derivatives was used (Malmberg, E., W., Hamilton, C., S., J. Am. Chem. Soc. & 0, 2415, (1948); Willenz, J. J. Chem Soc., 1955, 2049). The reaction includes acid-catalyzed attack of amino-compounds on mesomeric keto-form of the aromatic hydroxy-derivatives. The reaction product with 1,4-diaminobutane was obtained with high yield and purified by extraction. The resulting DDAO amino-derivative was converted to corresponding isothiocyanate (ITC) by treatment with thiocarbonyldiimidazole followed by incubation with trifluoroacetic acid (FIG. 2). Obtained &-aminoDDAO derivative was used to label antifungal drugs posaconazole and caspofungin (FIG. 3). Caspofungin was derivatized by the ITC in single-step reaction as one of the drugs two aliphatic amino groups. To introduce DDAO fluorescent label in posaconazole molecule the drug was first acylated at hydroxyl group by succinic anhydride in DMSO in the presence of nucleophilic catalyst, N-methylimidazole (FIG. 4). The resulting product was converted to an activated ester by incubation with 4-nitrophenole and DCC. This synthetic intermediate was introduced in reaction with 1,4-diaminobutyl-DDAO compound to yield the final product, which was purified using preparative TLC.

Light Absorption and Fluorescent Spectra of 7-aminoDDAO, Caspofungin-DDAO, and Posaconazole-DDAO Derivatives.

Modification of DDAO resulted in detectable blue shift of the light absorption maximum (653 nm and 673 nm correspondingly). The molar extinction of 7-aminoDDAO (55 000 M-1 cm-1) was determined by the attachment of reference chromophores with known molar absorptivity. Light absorption spectra of the labeled caspofungin and posaconazole derivatives were close to superposition of those for the 7-(4-aminobutyl)aminoDDAO and the corresponding drugs. Fluorescence spectra (FIG. 6B) of 7-(4-aminobutyl)aminoDDAO exhibited blue shift compared to ionized form of DDAO (FIG. 6A). Thus, excitation and emission maxima for DDAO were 653 nm and 660 nm correspondingly, while for 7-(4-aminobutyl)amino-DDAO they shifted to 671 nm and 679 nm correspondingly. Increasing content of the organic solvent (MeOH) resulted in enhancement of the light emission and characteristic change in excitation. Thus substitution 50% methanol for water did not affect the shape of the excitation spectrum for 7-aminoDDAP, but increased the light emission ca. 2.5 fold. Placing the compound in 100% MeOH resulted in dramatic change of the excitation spectrum profile shifting the maximum from 670 nm to 620 nm, while only slightly shifting emission maximum from 680 to 670 nm. Notably, the light emission intensity dropped 1.7 fold. Remarkably, the shape of the excitation spectrum curve for ionized form of DDAO was the same in 50% and 100% methanol. Also, in contrast to 7-aminoDDAO 1.3 fold increase in the emission was observed in 100% methanol comparing to 50% methanol.

In Vivo Use of Caspofungin-DDAO Derivative for Imaging of Fungal Infections.

Mice were infected via intravenous inoculation with 5*10⁵ CFU of wild type Candida albicans and an infection that occurs most prominently in the kidneys. After 48 hours post infection, a fixed concentration of 0.12 ug/mL of CSF-DDAO) is added via tail vein injection at 0, 2, 4 and 8 hours to assess the optimal time for visualization of the infection. At each time point, the mice were imaged in a non-invasive whole-body animal imaging system to detect fluorescence energy. Animals infected with Candida albicans show proliferation of the fungal infection in the kidneys after 48 hours. The addition of CSF probe resulted in progressive labeling of cells in the target organs over time, as determined by whole body imaging. Maximum labeling occurred at 8 hours. CSF-DDAO did not accumulate in the kidneys in the absence of infection.

All publications, cited in this disclosure are incorporated by reference in their entireties. The citation of any references herein is not an admission that such references are prior art to the present invention.

The embodiments within the specification provide an illustration of embodiments of the invention and should not be construed to limit the scope of the invention. The skilled artisan readily recognizes that many other embodiments are encompassed by the invention. Those skilled in the art will recognize, or be able to ascertain using no more than routine experimentation, many equivalents to the specific embodiments of the invention described herein. Such equivalents are intended to be encompassed by the following embodiments.

1. A method to detect a fungus in a subject comprising administering to said subject a targeting agent wherein said targeting agent comprises an antifungal drug covalently bound to a detectable label, and detecting said targeting agent, wherein said antifungal drug is selected from the group consisting of a polyene, an azole and an echinocandin.

2. The method of claim 1, wherein said detectable label is selected from the group consisting of a fluorescent label, a radioactive isotope, and a contrast agent.

3. The method of claim 2 wherein said detectable label is a fluorescent label.

4. The method of claim 3 wherein said fluorescent label is boron-dipyrromethene (BODIPY), 7-hydroxy-9H-(1,3-dichloro-9,9-dimethylacridin-2-one)(DDAO), 7-amino-9H-(1,3-dichloro-9,9-dimethylacridin-2-one)(7-aminoDDAO), or a derivative thereof.

5. A method to detect a fungus in a subject comprising administering to said subject a targeting agent wherein said targeting agent comprises an antifungal drug covalently bound to a detectable label, and detecting said targeting agent, wherein said detectable label is selected from the group consisting of boron-dipyrromethene (BODIPY), 7-hydroxy-9H-(1,3-dichloro-9,9-dimethylacridin-2-one)(DDAO), 7-amino-9H-(1,3-dichloro-9,9-dimethylacridin-2-one)(7-aminoDDAO), or a derivative thereof.

6. The method of claim 1 or 5, wherein said antifungal drug is selected from the group consisting of natamycin, rimocidin, filipin, nystatin, amphotericin B, candicin, miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, abafungin, terbinafine, amorolfine, naftifine, butenafine, anidulafungin, caspofungin, and micafungin.

7. The method of claim 1, wherein said targeting agent is caspofungin-7aminoDDAO.

8. The method of claim 1 or 5, wherein said subject is administered a pretreatment antifungal drug prior to the administration of said targeting agent, wherein said pretreatment antifungal drug and said the antifungal drug of said targeting agent are the same antifungal drug.

9. The method of claim 8, wherein said antifungal drug is caspofungin.

10. The method of claim 1 or 5, wherein said subject is administered a pretreatment antifungal drug prior to the administration of said targeting agent, wherein said pretreatment antifungal drug and the antifungal drug in said targeting agent are not the same antifungal drug and wherein said pretreatment antifungal drug does not bind to the same target as the antifungal drug in said targeting agent.

11. The method of claim 10, wherein said pretreatment antifungal drug is posaconazole, and the antifungal drug in said targeting agent is caspofungin.

12. The method of claim 1 wherein the targeting agent is detected using an imaging device.

13. The method of claim 12 wherein said imaging device is selected from the group consisting of an x-ray imaging device, an infrared imaging device, fluorescent imaging device, nuclear magnetic resonance imaging device, magnetic resonance spectroscopy device, and a positron emission tomography device.

14. The method of claim 1 or 5, wherein said fungus is selected from the group consisting of Candida albicans, Candida glabrata, Candida parapsilosis, Candida krusei, Aspergillus fumigatus, Aspergillus niger, Aspergillus flavus, Cryptococcus neoformans, Scedosporium apiospermum, Zygomycetes, Histoplasma capsulatum, Coccidioides immitis, Paracoccidiioides brassiliensis and Blastomyces dermatitidis.

15. A targeting agent comprising an antifungal agent conjugated directly to a detectable label, wherein said antifungal drug is selected from the group consisting of antifungal drug is selected from the group consisting of a polyene, an azole and an echinocandin.

16. A targeting agent comprising an antifungal agent conjugated directly to a detectable label, wherein said detectable label is selected from the group consisting of boron-dipyrromethene (BODIPY), 7-hydroxy-9H-(1,3-dichloro-9,9-dimethylacridin-2-one)(DDAO), 7-amino-9H-(1,3-dichloro-9,9-dimethylacridin-2-one)(7-aminoDDAO), or a derivative thereof.

17. The targeting agent of claim 15 or 16, wherein said antifungal agent is caspofungin or posacozole.

18. The targeting agent of claim 15, wherein said detectable label is boron-dipyrromethene, 7-hydroxy-9H-(1,3-dichloro-9,9-dimethylacridin-2-one), 7-amino-9H-(1,3-dichloro-9,9-dimethylacridin-2-one) or a derivative thereof.

19. The targeting agent of claim 15, wherein said antifungal agent is caspofungin and said label is 7-amino-9H-(1,3-dichloro-9,9-dimethylacridin-2-one).

20. The targeting agent of claim 15, wherein said antifungal agent is posacozole and said label is 7-amino-9H-(1,3-dichloro-9,9-dimethylacridin-2-one).

21. A kit for detecting a fungus in a biological sample or a subject comprising the targeting agent of claim 15 or 16, and instructions for use.

22. The targeting agent of claim 15 wherein the detectable label is a fluorescent label.

23. A method to detect a fungus in a subject comprising administering to said subject a targeting agent wherein said targeting agent comprises an antifungal drug covalently bound to a detectable label, and detecting said targeting agent.

24. A targeting agent comprising an antifungal agent conjugated directly to a detectable label.
